(19) United States
(12) Patent Application Publication
(10) Pub. No.: US 2014/0206968 A1
(43) Pub. Date: Jul. 24, 2014

(54) Ultrasonic Sensor and Method of Operating the Same

(71) Applicant: FLEXTRONICS AP, LLC, San Jose, CA (US)

(72) Inventor: Lior Shtram, Tel Aviv (IL)

(73) Assignee: FLEXTRONICS AP, LLC, San Jose, CA (US)

(21) Appl. No.: 13/749,548

(22) Filed: Jan. 24, 2013

(51) Int. Cl. A61M 5/172; A61B 5/145; G01S 15/89

(52) U.S. Cl. CPC A61M 5/1723; G01S 15/89; A61B 5/14532
USPC 600/365; 367/87; 604/65

(57) ABSTRACT

A system including a host device and a measurement device ultrasonically coupled to the host device over a physical medium and method of operating the same has been introduced herein. In one embodiment, the system includes the host device including a first ultrasonic communicator configured to generate an ultrasonic command to measure a parameter of an object, a physical medium coupled to the host device and the measurement device coupled to the physical medium. The measurement device includes a second ultrasonic communicator configured to receive the ultrasonic command via the physical medium, and a sensor configured to measure a parameter of the object in response to the ultrasonic command to provide a sensed parameter, the second ultrasonic communicator being configured to transmit the sensed parameter to the host device via the physical medium.

### TECHNICAL FIELD

The present invention is directed, in general, to ultrasonic communication and, more specifically, to an ultrasonic sensor and method of operating the same.

### BACKGROUND

In medical and other applications, a disposable physical medium such as a flexible plastic tube is generally used to enable a mechanical function to be performed such as a surgical procedure or to transport an injectable fluid such as a drug. When additional functionality is included in the physical medium such as an electronic sensor or actuator positioned at a distal end thereof, wiring is embedded within or even positioned external to the medium envelope to provide power for and communication with the distantly positioned electronic sensor or actuator. Embedded wiring, however, can mechanically interfere with the basic mechanical functionality of the physical medium. A separate wiring connection added in parallel with and separate from the physical medium adds other mechanical issues. Both approaches raise questions of patient safety or added cost to provide electrical safety isolation when a direct wired electrical connection is provided between a patient and host electrical equipment.

An alternative method of communicating with an electronic sensor or actuator positioned at a distal end of a disposable physical medium employs radio frequency (“RF”) signaling. However, RF signals can be strongly and unpredictably attenuated by intervening structures such as by an object such as a patient's body and by a metallic apparatus that might be uncontrollably positioned between an RF transmitter and receiver. Another disadvantage of RF signaling is electromagnetic interference to other nearby equipment produced by the signaling itself.

Accordingly, what is needed in the art is a system and method to communicate with a sensor or actuator located at a remote end of a physical medium such as a disposable physical medium that avoids disadvantages of present systems.

### SUMMARY OF THE INVENTION

Technical advantages are generally achieved, by advantageous embodiments of the present invention, including a system including a host device and a measurement device ultrasonically coupled to the host device over a physical medium and method of operating the same has been introduced herein. In one embodiment, the system includes the host device including a first ultrasonic communicator configured to generate an ultrasonic command to measure a parameter of an object, a physical medium coupled to the host device and the measurement device coupled to the physical medium. The measurement device includes a second ultrasonic communicator configured to receive the ultrasonic command via the physical medium, and a sensor configured to measure a parameter of the object in response to the ultrasonic command to provide a sensed parameter, the second ultrasonic communicator being configured to transmit the sensed parameter to the host device via the physical medium.

The foregoing has outlined rather broadly the features and technical advantages of the present invention in order that the detailed description of the invention that follows may be better understood. Additional features and advantages of the invention will be described hereinafter, which form the subject of the claims of the invention. It should be appreciated by those skilled in the art that the conception and specific embodiment disclosed may be readily utilized as a basis for modifying or designing other structures or processes for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, reference is now made to the following descriptions taken in conjunction with the accompanying drawings, in which:

FIG. 1 illustrates a block diagram of an embodiment of a system formed with a host device and a measurement device coupled to the host device by a physical medium constructed according to the principles of the present invention;

FIG. 2 illustrates a block diagram of an embodiment of a host device formed with an insulin pump controlled by host device functional elements and coupled to a measurement device such as a cannula constructed according to the principles of the present invention;

FIG. 3 illustrates a graphical representation of an embodiment of a host device formed with wound care equipment controlled by host device functional elements and coupled over a physical medium to a measurement device positioned on an object constructed according to the principles of the present invention;

FIG. 4 illustrates a block diagram of an embodiment of a host device coupled over a physical medium to a measurement device constructed according to the principles of the present invention;

FIG. 5 illustrates a graphical representation showing further structure of the host device illustrated in FIG. 4 constructed according to the principles of the present invention;

FIG. 6 illustrates a block diagram of an embodiment of a measurement device constructed according to the principles of the present invention; and

FIG. 7 illustrates a block diagram of an embodiment of a host device constructed according to the principles of the present invention.

Corresponding numerals and symbols in the different figures generally refer to corresponding parts unless otherwise indicated, and may not be redescribed in the interest of brevity after the first instance. The FIGUREs are drawn to illustrate the relevant aspects of exemplary embodiments.

### DETAILED DESCRIPTION OF ILLUSTRATIVE EMBODIMENTS

The making and using of the present exemplary embodiments are discussed in detail below. It should be appreciated, however, that the present invention provides many applicable inventive concepts that can be embodied in a wide variety of specific contexts. The specific embodiments discussed are merely illustrative of specific ways to make and use the invention, and do not limit the scope of the invention.

The present invention will be described with respect to exemplary embodiments in a specific context, namely, an ultrasonic communicator in a host device configured to communicate bidirectionally over a physical medium such as a disposable and detachable functional physical medium with an ultrasonic communicator in a measurement device. While the principles of the present invention will be described in the environment of a medical application, any application that may benefit from ultrasonic communication over a physical medium such as a plastic tube or a surgically controlled shaft is well within the broad scope of the present invention.

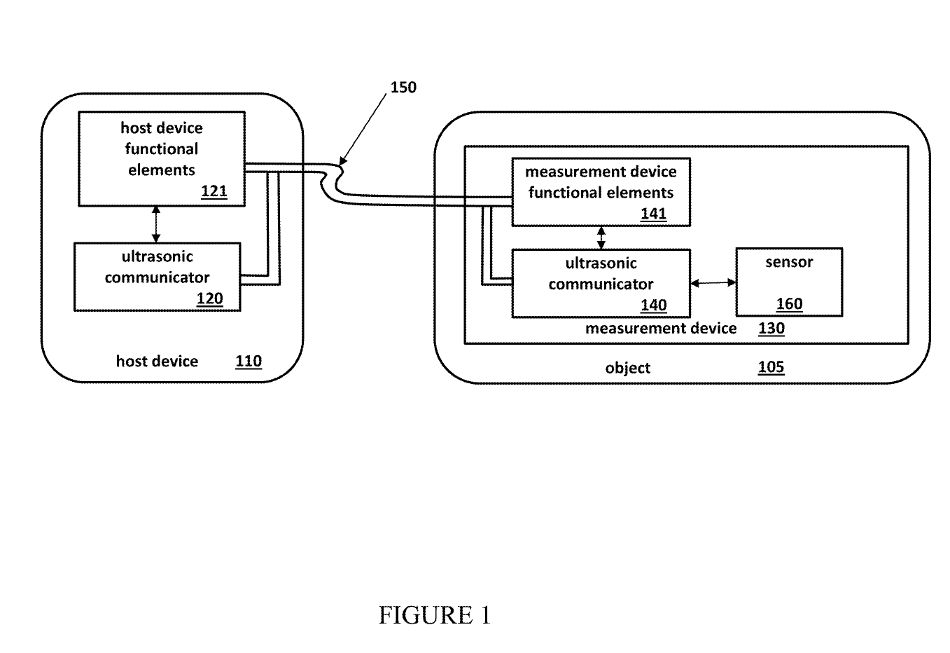

Referring initially to FIG. 1, illustrated is a block diagram of an embodiment of a system formed with a host device 110 and a measurement device 130 (e.g., a disposable measurement device) coupled to the host device 110 by a physical medium 150 constructed according to the principles of the present invention. The measurement device 130 can be attached to or embedded into an object 105 such as a patient or other physical object. The host device 110 includes host device functional elements 121 that perform a task in conjunction with the physical medium 150 such as management of a patient's blood glucose level or performing a physical procedure such as a surgical task. The host device 110 is coupled to and controls the measurement device 130 by the physical medium 150 such as a disposable and detachable flexible plastic tube, a mechanical shaft, a disposable and detachable catheter, etc.

The host device 110 includes an ultrasonic communicator 120 (a first ultrasonic communicator) coupled to the physical medium 150 and to the host device functional elements 121. The ultrasonic communicator 120 is configured to transmit and receive ultrasonic signals over the physical medium 150 to and from an ultrasonic communicator 140 (a second ultrasonic communicator) located in the measurement device 130 at a distal end of the physical medium 150. The ultrasonic communicator 140 is formed with a piezoelectric element and is coupled to and transmits an ultrasonic signal to control measurement device functional elements 141. Thus, the measurement device 130 operates in response to an ultrasonic control signal (an ultrasonic command) transmitted by the host device 110 to measure a parameter or the like. The ultrasonic communicator 140 is also coupled to a sensor 160 that can detect/measure an environmental characteristic (a sensed parameter) at the measurement device 130 in response to control signals transmitted by the host device 110.

The measurement device 130 and/or the physical medium 150 may be disposable and detachable components. By employing ultrasonic signals to enable the host device functional elements 121 to communicate with the measurement device 130, issues of electromagnetic interference and safety issues related to electrically coupling the object 105 (e.g., a patient) to a host electrical system are avoided.

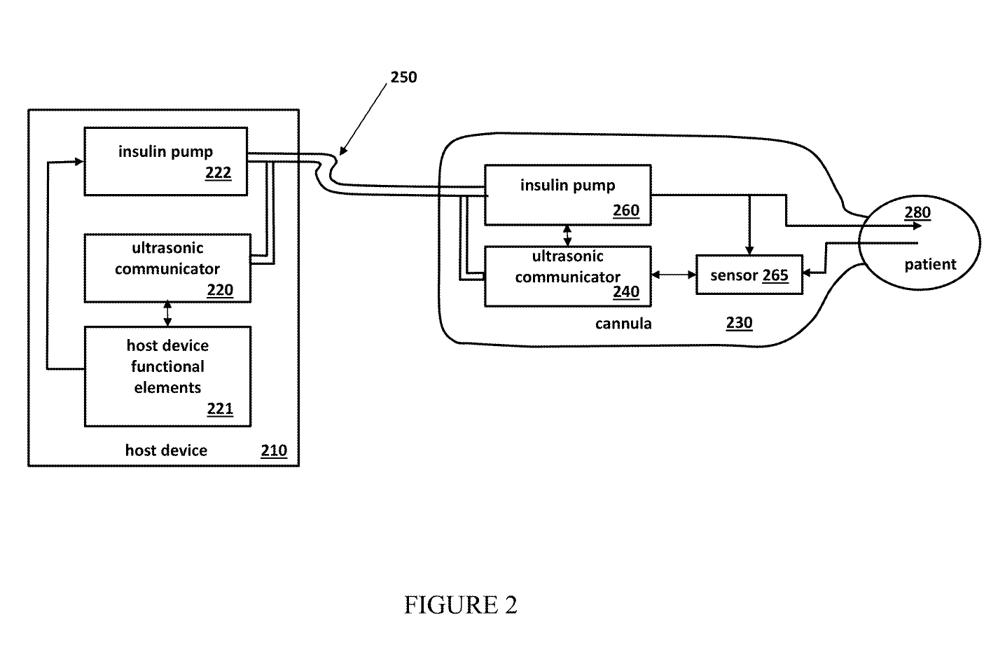

Turning now to FIG. 2, illustrated is a block diagram of an embodiment of a host device 210 formed with an insulin pump 222 controlled by host device functional elements 221 of the host device 210 and coupled to a measurement device such as a cannula 230 (e.g., a disposable and detachable cannula) constructed according to the principles of the present invention. The host device 210 in conjunction with the cannula 230 is configured to controllably administer a fluid such as an insulin dose to an object such as patient 280. A physical medium such as a plastic tube 250 that couples the host device 210 to the cannula 230 conveys insulin from the insulin pump 222 (a first dispenser in the host device 210) to an insulin pump 260 (a second dispenser in the cannula 230) that in turn administers a controlled insulin dose that is controlled by the host device functional elements 221. A sensor 265 in the cannula 230 is an electronic element that senses a volume of the insulin dose delivered by the insulin pump 260 in the cannula 230. An ultrasonic communicator 240, a bidirectional ultrasonic communicator, is configured to report with an ultrasonic signal the sensed volume of the insulin dose back to an ultrasonic communicator 220 in the host device 210.

The sensor 265 may sense a characteristic of the patient 280 such as a chemical marker in a blood sample, a blood glucose level, or the occurrence of bleeding. Thus, the sensor 265 measures a characteristic of the patient 280 such as delivered insulin volume or blood glucose and communicates data to the ultrasonic communicator 240 (in the cannula 230) that transmits the data back to the ultrasonic communicator 220 in host device 210.

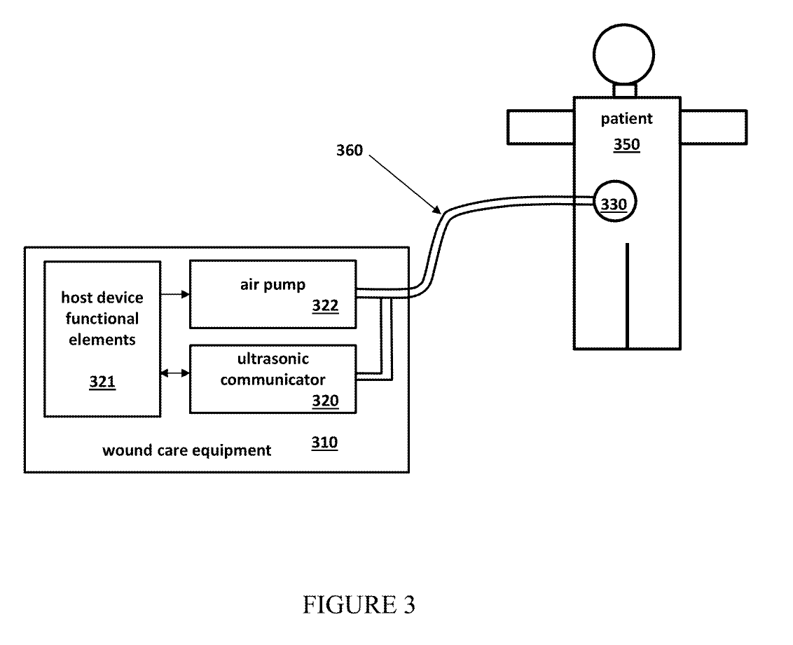

Turning now to FIG. 3, illustrated is a graphical representation of an embodiment of a host device formed with wound care equipment 310 including an air pump 322 controlled by host device functional elements 321 and coupled over a physical medium such as disposable and detachable flexible plastic tube 360 to a measurement device such as a negative pressure wound dressing 330 positioned on an object such as patient 350 constructed according to the principles of the present invention. The host device functional elements 321 control negative pressure in the negative pressure wound dressing 330 that forms an air seal on patient skin by sensing air pressure with a sensor in the wound dressing. The negative pressure wound dressing 330 transmits the sensed pressure ultrasonically to an ultrasonic communicator 320 in the wound care equipment 310 and ultimately to host device functional elements 321.

The sensor in the negative pressure wound dressing 330 may measure an environmental characteristic such as a chemical marker or other indicator of the wound condition and transmits the sensed characteristic to the wound care equipment 310 to alert a physician. In the embodiment as illustrated in FIG. 3, the wound care equipment 310 generates an alarm for a physician or other attendant in response to data transmitted by the negative pressure wound dressing 330.

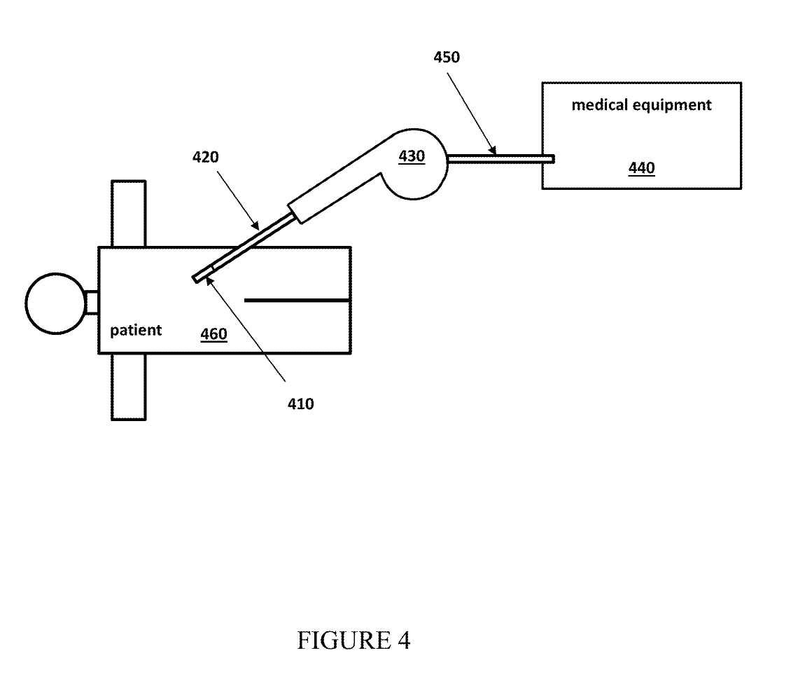

Turning now to FIG. 4, illustrated is a block diagram of an embodiment of a host device 430 formed with mechanical/electromechanical surgical elements coupled over a physical medium such as a laparoscopic/endoscopic tube or RF catheter 420 to a measurement device 410 for application to an object such as patient 460 constructed according to the principles of the present invention. The measurement device 410 is located at a distal end of the laparoscopic/endoscopic tube or RF catheter 420 and is configured to communicate ultrasonically with the host device 430 over the laparoscopic/endoscopic tube or RF catheter 420. The host device 430 obtains environmental measurements (a sensed parameter) from the measurement device 410 in response to an ultrasonic signal or command transmitted to the measurement device 410 by the host device 430 such as, without limitation, a characteristic of tissue that the laparoscopic/endoscopic tube contacts, a tissue thickness, or a biometric indicator of tissue status.

The host device 430 may control an RF tissue heater at a distal end of laparoscopic/endoscopic tube or RF catheter 420 employing an ultrasonic signal transmitted over the laparoscopic/endoscopic tube or RF catheter 420. The measurement device 410 transmits a sensed environmental characteristic back to the host device 430 in response to an ultrasonic command transmitted to the measurement device 410 by the host device 430. The host device 430 is coupled by a cable 450 or other attachment means to medical equipment 440 such as a monitor or a recording device. The host device 430 can communicate with the medical equipment 440 by a wireless signal or by a signal transmitted over a wired path.

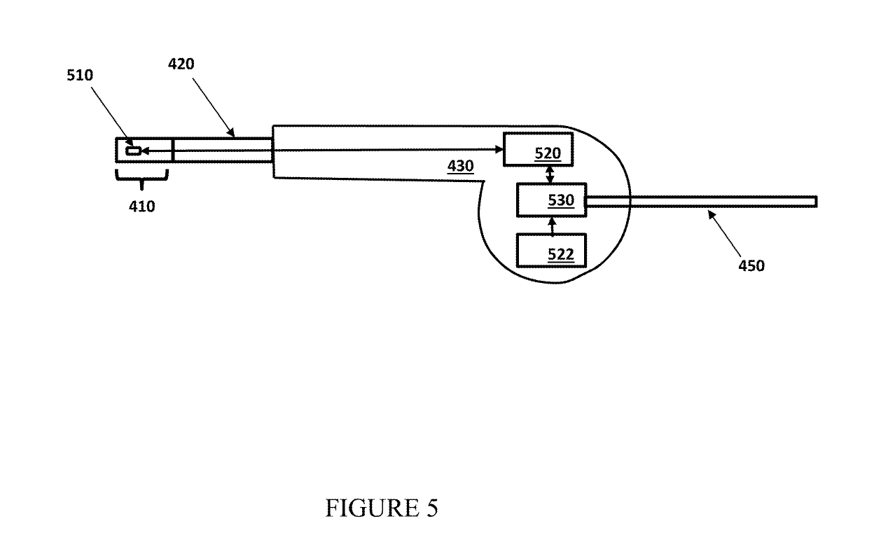

Turning now to FIG. 5, illustrated is a graphical representation showing further structure of the host device 430 illustrated in FIG. 4 constructed according to the principles of the present invention. The host device 430 is adapted for use as a hand-held surgical tool and is coupled to the laparoscopic/endoscopic tube or RF catheter 420. By including a disposable and detachable laparoscopic/endoscopic tube or RF catheter 420 at an end of the host device 430, the need to dispose of or maintain a more complex and costly host device 430 is eliminated. The measurement device 410 is located at a distal end of the laparoscopic/endoscopic tube or RF catheter 420 and includes a mechanical actuator(s) configured to perform a portion of a laparoscopic/endoscopic or RF-ablative surgical procedure. The measurement device 410 includes a sensor and ultrasonic communicator 510 as described previously hereinabove that enables the measurement device 410 to communicate ultrasonically with an ultrasonic communicator 520 in the host device 430. In this manner, the need to run sensor wiring through the laparoscopic/endoscopic tube or RF catheter 420 which can interfere with mechanical elements thereof is avoided.

The host device 430 includes host device functional elements 530 coupled to the ultrasonic communicator 520 to enable the host device functional elements 530 to control and obtain data from the measurement device 410 at the distal end of the laparoscopic/endoscopic tube or RF catheter 420 to enable the host device 430 to be used to perform surgical procedures.

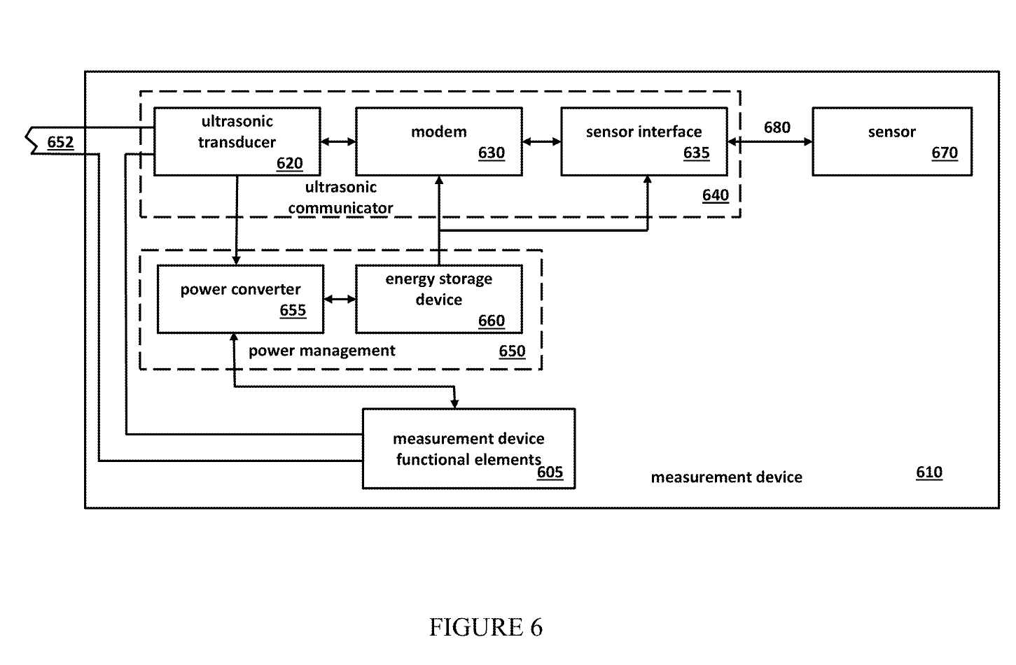

The host device 430 can be coupled to the medical equipment 440 (see FIG. 4) by the cable 450. The cable 450 can be employed to provide power and control signals to the host device 430. In an embodiment, a wireless process such as a Wi-Fi™ signal can be employed for the host device 430 to communicate with the medical equipment 440. The host device 430 includes a power management block 522 that provides electrical power for the host device functional elements 530 and the ultrasonic communicator 520. Further structure of the power management block 522 is described hereinbelow with reference to FIG. 6.

Turning now to FIG. 6, illustrated is a block diagram of an embodiment of a measurement device 610 constructed according to the principles of the present invention. The measurement device 610, which can be a disposable measurement device, is formed with measurement device functional elements 605 that are coupled to a physical medium 652 such as a tube, cannula, or shaft. An example of a measurement device functional element 605 is a downstream pump configured to deliver a controllable volume of a drug such as insulin to a patient in response to an ultrasonic command received by the measurement device 605 from a host device. The measurement device 610 is formed with an ultrasonic communicator 640 coupled to the physical medium 652.

The ultrasonic communicator 640 is formed with an ultrasonic transducer 620 that is constructed with a piezoelectric element that is acoustically coupled to the physical medium 652. The ultrasonic transducer 620 is coupled to a modem 630 that converts signals to and from the ultrasonic transducer 620 to an electrical format usable by a sensor interface 635. The sensor interface 635 is coupled over an electrical connection 680 to a sensor 670. Example environmental characteristics that can be sensed by the sensor 670 are environmental characteristics such as a serum glucose level, a chemical marker, a temperature, and a tissue thickness. The measurement device 610 can be constructed to controllably dispense a fluid such as a controlled volume of a drug such as insulin to an object such as a patient in response to an ultrasonic command received over the physical medium 652 from a host device.

A power management block 650 provides a source of electric energy for the several electrical components in the measurement device 610. The power management block 650 is formed with a power converter 655 and an energy storage device 660. The power converter 655 is electrically coupled to the ultrasonic transducer 620, which converts ultrasonic energy (from an ultrasonic command) transmitted along the physical medium 652 by a host device into an electrical form. Electrical energy is conditioned by the V 655 to be stored in an energy storage device 660, which can be a rechargeable battery or a capacitor such as an electrolytic or chemical capacitor. A power source is thus provided for the measurement device 610 that does not depend on a wired electrical connection to a host device. In this manner the need to meet safety standards for an electrical device that can be placed in contact with a patient and that is powered by a high-level power source such as alternating current (“ac”) mains is avoided.

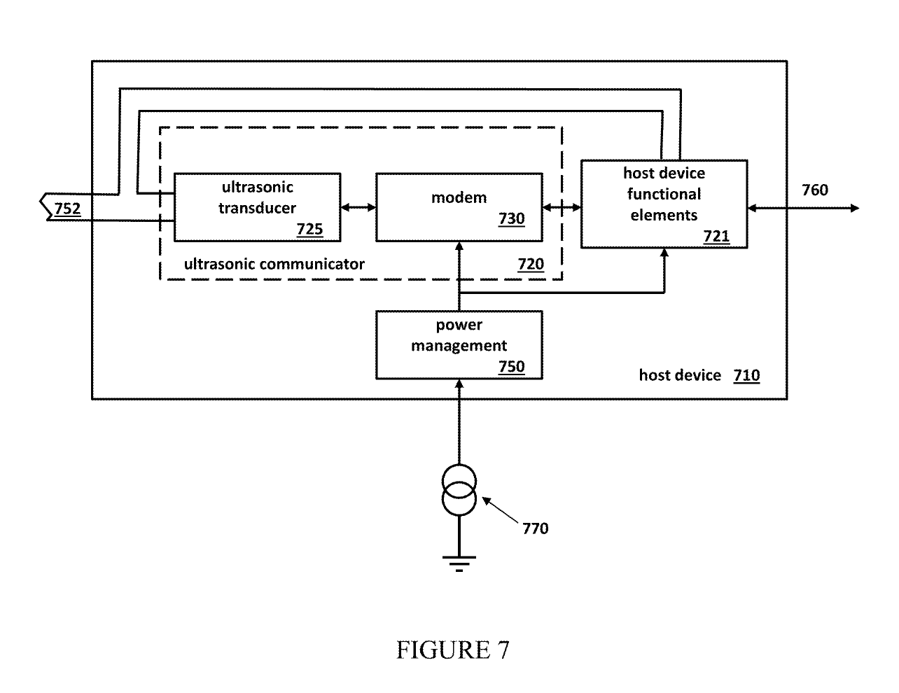

Turning now to FIG. 7, illustrated is a block diagram of an embodiment of a host device 710 constructed according to the principles of the present invention. The host device 710 is formed with host device functional elements 721 that are coupled to a physical medium 752 that enable execution of an intended task such as management of a patient's blood glucose level or performing a physical procedure such as a surgical task. The host device 710 is formed with an ultrasonic communicator 720 that includes an ultrasonic transducer 725 that is acoustically coupled to the physical medium 752 to receive ultrasonic signals conductive along the physical medium 752. The ultrasonic transducer 725 is coupled to a modem 730 that converts signals produced by and coupled to the ultrasonic transducer 725 to an electrical format usable by a sensor interface of a measurement device (e.g., the sensor interface 635 of the measurement device 610 of FIG. 6). The modem 730 is coupled to the host device functional elements 721 to enable the host device functional elements 721 to receive signals from and control elements in a measurement device (e.g., the measurement device 610 of FIG. 6) coupled to a distal end of the physical medium 752. The host device functional elements 721 can be coupled by a cable 760 or other attachment means to external medical equipment such as the medical equipment 440 illustrated hereinabove in FIG. 4. The host device 710 can communicate with the medical equipment by wireless means or by a wired path.

The host device 710 can be configured to provide a source of a fluid that can be a drug such as insulin that is conveyed over the physical medium 752 to a downstream controllable fluid dispenser such as a pump located within a measurement device for delivery to an object such as a patient. A power management block 750 is coupled to an electrical energy source such as ac mains 770 and provides the necessary power conversion means to provide electrical power for the several components of the host device 710 such as the modem 730 and the host device functional elements 721.

Thus, a system including a host device and a measurement device ultrasonically coupled to the host device over a physical medium (e.g., a tube such as a flexible plastic tube and a laparoscopic device) and method of operating the same has been introduced herein. In one embodiment, the system includes the host device including a first ultrasonic communicator configured to generate an ultrasonic command to measure a parameter of an object, a physical medium coupled to the host device and the measurement device coupled to the physical medium. The measurement device includes a second ultrasonic communicator configured to receive the ultrasonic command via the physical medium, and a sensor configured to measure a parameter of the object in response to the ultrasonic command to provide a sensed parameter, the second ultrasonic communicator being configured to ultrasonically transmit the sensed parameter to the host device via the physical medium.

The measurement device may be located within and at a distal end of the physical medium. The measurement device may include a piezoelectric element configured to employ the ultrasonic command to provide a power source for the measurement device, the power source being employed to recharge a battery in the measurement device. The physical medium may be a disposable tube and the measurement device is a disposable measurement device. In an embodiment, the object is a patient and the host device further includes a first dispenser configured to dispense a fluid (e.g., a drug) for transmission to a second dispenser within the measurement device via the physical medium for delivery to the patient. In another embodiment, the object is a patient and the parameter is a serum glucose level of the patient.

The system or related method may be implemented as hardware (embodied in one or more chips including an integrated circuit such as an application specific integrated circuit), or may be implemented as software or firmware for execution by a processor (e.g., a digital signal processor) in accordance with memory. In particular, in the case of firmware or software, the exemplary embodiment can be provided as a computer program product including a computer readable medium embodying computer program code (i.e., software or firmware) thereon for execution by the processor.

Program or code segments making up the various embodiments may be stored in the computer readable medium. For instance, a computer program product including a program code stored in a computer readable medium (e.g., a non-transitory computer readable medium) may form various embodiments. The “computer readable medium” may include any medium that can store or transfer information. Examples of the computer readable medium include an electronic circuit, a semiconductor memory device, a read only memory (“ROM”), a flash memory, an erasable ROM (“EROM”), a floppy diskette, a compact disk (“CD”)-ROM, and the like.

Those skilled in the art should understand that the previously described embodiments of a system and related methods of forming the same are submitted for illustrative purposes only. A system as described hereinabove may also be applied in other applications in addition to medical applications.

Also, although the present invention and its advantages have been described in detail, it should be understood that various changes, substitutions and alterations can be made herein without departing from the spirit and scope of the invention as defined by the appended claims. For example, many of the processes discussed above can be implemented in different methodologies and replaced by other processes, or a combination thereof.

Moreover, the scope of the present application is not intended to be limited to the particular embodiments of the process, machine, manufacture, composition of matter, means, methods, and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A system, comprising:
a host device including a first ultrasonic communicator configured to generate an ultrasonic command to measure a parameter of an object;
a physical medium coupled to said host device; and
a measurement device coupled to said physical medium, comprising:
a second ultrasonic communicator configured to receive said ultrasonic command via said physical medium, and
a sensor configured to measure said parameter of said object in response to said ultrasonic command to provide a sensed parameter, said second ultrasonic communicator being configured to transmit said sensed parameter to said host device via said physical medium.

2. The system as recited in claim 1 wherein said sensed parameter is transmitted ultrasonically to said host device via said physical medium.

3. The system as recited in claim 1 wherein said physical medium is a tube.

4. The system as recited in claim 1 wherein said physical medium comprises a flexible plastic tube.

5. The system as recited in claim 1 wherein said physical medium comprises a laparoscopic device.

6. The system as recited in claim 1 wherein said object is a patient and said host device further comprises a first dispenser configured to dispense a fluid for transmission to a second dispenser within said measurement device via said physical medium for delivery to said patient.

7. The system as recited in claim 6 wherein said fluid is a drug.

8. The system as recited in claim 1 wherein said measurement device is located within and at a distal end of said physical medium.

9. The system as recited in claim 1 wherein said measurement device comprises a piezoelectric element configured to employ said ultrasonic command to provide a power source for said measurement device.

10. The system as recited in claim 9 wherein said power source is employed to recharge a battery in said measurement device.

11. The system as recited in claim 1 wherein said object is a patient and said parameter is a serum glucose level of said patient.

12. The system as recited in claim 1 wherein said physical medium is a disposable tube and said measurement device is a disposable measurement device.

13. A method, comprising:
generating an ultrasonic command to measure a parameter of an object with a host device;
receiving said ultrasonic command via a physical medium;
measuring said parameter of said object in response to said ultrasonic command to provide a sensed parameter with a measurement device; and
transmitting said sensed parameter to said host device via said physical medium.

14. The method as recited in claim 13 further comprising ultrasonically transmitting said sensed parameter to said host device via said physical medium.

15. The method as recited in claim 13 wherein said object is a patient and further comprising dispensing a fluid from said host device to said measurement device via said physical medium for delivery to said patient.

16. The method as recited in claim 13 wherein said measurement device is located within and at a distal end of said physical medium.

17. The method as recited in claim 13 further comprising providing a power source for said measurement device in accordance with a piezoelectric element thereof.

18. The method as recited in claim 17 further comprising recharging a battery in said measurement device with said power source.

19. The method as recited in claim 13 wherein said object is a patient and said parameter is a serum glucose level of said patient.

20. The method as recited in claim 13 wherein said physical medium is a disposable tube and said measurement device is a disposable measurement device.
